(19) United States
(12) Patent Application Publication
(10) Pub. No.: US 2026/0191537 A1
(43) Pub. Date: Jul. 9, 2026

(54) TIBIAL ALIGNMENT JIG INSTRUMENT AND METHOD OF USING THE SAME IN AN ORTHOPAEDIC SURGICAL KNEE PROCEDURE

(71) Applicant: DePuy Ireland Unlimited Company, Ringaskiddy (IE)

(72) Inventors: Nicholas A. Miltner, Fort Wayne, IN (US); Jeremiah M. Lewis, Warsaw, IN (US); James Brooks, Burley in Wharfedale (GB); Jason M. Chavarria, Warsaw, IN (US)

(21) Appl. No.: 19/417,737

(22) Filed: Dec. 12, 2025

Related U.S. Application Data
(60) Provisional application No. 63/741,632, filed on Jan. 3, 2025.

(51) Int. Cl. A61B 17/15

(52) U.S. Cl. CPC A61B 17/157

(57) ABSTRACT

A tibial alignment jig includes a proximal tibial jig assembly having a varus/valgus adjustment arm and a posterior slope adjustment arm. A tibial stylus instrument includes a pair of styluses configured to contact the tibial plateaus of a patient's tibia. The tibial alignment jig and the tibial stylus instrument may be operated in either a mechanical alignment mode of operation or a kinematic alignment mode of operation. Methods of surgically preparing a patient's tibia are also disclosed.

This application claims priority to U.S. Provisional Patent Application Ser. No. 63/741,632 which was filed on Jan. 3, 2025, which is hereby incorporated by reference in its entirety.

### CROSS REFERENCE

Cross reference is made to copending U.S. Patent application Ser. No. XX/XXX,XXX entitled “TIBIAL STYLUS INSTRUMENT AND METHOD OF USING THE SAME IN AN ORTHOPAEDIC SURGICAL KNEE PROCEDURE” (Attorney Docket No. 265280-431231, DEP7190USNP 2), which is assigned to the same assignee as the present application, filed concurrently herewith, and hereby incorporated by reference in its entirety.

### TECHNICAL FIELD

The present disclosure relates generally to orthopaedic surgical instruments and, more particularly, to surgical instruments used to resect a patient's tibia.

### BACKGROUND

Joint arthroplasty is a well-known surgical procedure by which a diseased and/or damaged natural joint is replaced by a prosthetic joint. For example, in a total knee arthroplasty surgical procedure, a patient's natural knee joint is partially or totally replaced by a prosthetic knee joint or knee prosthesis. To facilitate the replacement of the natural joint with the prosthesis, orthopaedic surgeons use a variety of orthopaedic surgical instruments such as, for example, saws, drills, reamers, rasps, broaches, cutting blocks, drill guides, milling guides, and other surgical instruments.

In total knee arthroplasty (TKA), the femur and tibia of the patient's knee are resected to create planar surfaces onto which a prosthetic femoral component and tibial component, respectively, are installed. Traditional TKA involves determining the resection planes based on a pre-determined angle as a function of mechanical alignment or by using a balanced approach that sets the resection planes based on ligament tension. More recently, kinematic alignment techniques involve determining the resection planes as a function of the native, pre-disease state of the patient's knee.

### SUMMARY

According to one aspect of the disclosure, an orthopaedic surgical instrument assembly for resecting a patient's tibia during an orthopaedic knee replacement procedure includes a tibial cutting block having a mediolaterally extending cutting slot, a proximal tibial jig assembly, and a distal tibial jig assembly. The proximal tibial jig assembly includes an extramedullary proximal rod and a block connector on a superior end of the proximal tibial jig assembly. The tibial cutting block is secured to the proximal tibial jig assembly with the block connector. The block connector pivots relative to the proximal rod about a mediolaterally extending pivot joint so as to adjust a posterior slope angle of the cutting slot. The block connector also pivots relative to the proximal rod about an anteroposteriorly extending pivot joint so as to adjust a varus/valgus angle of the cutting slot. The distal tibial jig assembly is secured to the proximal tibial jig assembly and includes an ankle clamp positioned on its inferior end.

In an example, the proximal tibial jig assembly further includes a varus/valgus adjustment arm. A superior end of the varus/valgus adjustment arm is coupled to the block connector and pivotally coupled to the proximal rod via the anteroposteriorly extending pivot joint such that rotation of the varus/valgus adjustment arm about the anteroposteriorly extending pivot joint adjusts the varus/valgus angle of the cutting slot.

In an example, an inferior end of the varus/valgus adjustment arm includes a locking grip that is operable to lock the varus/valgus adjustment arm in a selected rotational position relative to the proximal rod.

In an example, the proximal tibial jig assembly further comprises an indexing plate coupled to the proximal rod, the indexing plate having a plurality of locking notches positioned along its length.

The locking grip of the varus/valgus adjustment arm engages one of the plurality of locking notches of the indexing plate to lock the varus/valgus adjustment arm in the selected rotational position relative to the proximal rod. The locking grip includes a grip and a cam that interacts with the grip to maintain the grip in its locked or unlocked position.

In another example, the proximal tibial jig assembly also includes a posterior slope adjustment arm. A superior end of the posterior adjustment arm is coupled to the block connector and pivotally coupled to the varus/valgus adjustment arm via the mediolaterally extending pivot joint such that rotation of the posterior slope adjustment arm about the mediolaterally extending pivot joint adjusts the posterior slope angle of the cutting slot.

In an example, an inferior end of the posterior slope adjustment arm includes a locking lever that is operable to lock the posterior slope adjustment arm in a selected rotational position relative to the proximal rod.

The block connector may be integrally formed in the superior end of the posterior slope adjustment arm. The block connector may also include a locking clip.

According to another aspect, a method of surgically preparing a patient's tibia during an orthopaedic surgical knee procedure includes securing a tibial cutting block to a proximal tibial jig assembly, with the tibial cutting block having a mediolaterally extending cutting slot. The method also includes securing a distal tibial jig assembly to the proximal tibial jig assembly. The distal tibial jig assembly has an ankle clamp. A varus/valgus adjustment arm of the proximal tibial jig assembly is rotated so as to adjust a varus/valgus angle of the cutting slot, and a posterior slope adjustment arm of the proximal tibial jig assembly is rotated so as to adjust a posterior slope angle of the cutting slot.

The varus/valgus adjustment arm of the proximal tibial jig assembly may be rotated so as to position the varus/valgus adjustment arm in a selected rotational position, and then a locking grip is operated to lock the varus/valgus adjustment arm in the selection rotational position.

The posterior slope adjustment arm of the proximal tibial jig assembly may be rotated so as to position the posterior slope adjustment arm in a selected rotational position, and then a locking lever is operated to lock the posterior slope adjustment arm in the selection rotational position.

The posterior slope adjustment arm may be pivoted relative to the varus/valgus adjustment arm.

The assembled proximal and distal tibial jig assemblies are positioned on the patient's tibia such that the ankle clamp is clamped to the patient's ankle and the tibial cutting block abuts the patient's proximal tibia.

According to another aspect, an orthopaedic surgical instrument assembly for resecting a patient's tibia during an orthopaedic knee replacement procedure includes a tibial cutting block having a mediolaterally extending cutting slot, a proximal tibial jig assembly, and a distal tibial jig assembly. The proximal tibial jig assembly has an extramedullary proximal rod, a varus/valgus adjustment arm pivotally coupled to the proximal rod via an anteroposteriorly extending pivot joint, and a posterior slope adjustment arm pivotally coupled to the varus/valgus adjustment arm via a mediolaterally extending pivot joint. A block connector is secured to a superior end of the posterior slope adjustment arm, with the tibial cutting block configured to be secured to the block connector. Rotation of the varus/valgus adjustment arm about the anteroposteriorly extending pivot joint adjusts a varus/valgus angle of the cutting slot, whereas rotation of the posterior slope adjustment arm about the mediolaterally extending pivot joint adjusts a posterior slope angle of the cutting slot. The distal tibial jig assembly is configured to be secured to the proximal tibial jig assembly and includes an ankle clamp positioned on its inferior end.

In an example, an inferior end of the varus/valgus adjustment arm includes a locking grip that is operable to lock the varus/valgus adjustment arm in a selected rotational position relative to the proximal rod.

In an example, the proximal tibial jig assembly further comprises an indexing plate coupled to the proximal rod, the indexing plate having a plurality of locking notches positioned along its length.

The locking grip of the varus/valgus adjustment arm engages one of the plurality of locking notches of the indexing plate to lock the varus/valgus adjustment arm in the selected rotational position relative to the proximal rod.

In an example, an inferior end of the posterior slope adjustment arm includes a locking lever that is operable to lock the posterior slope adjustment arm in a selected rotational position relative to the proximal rod.

The block connector may be integrally formed in the superior end of the posterior slope adjustment arm. The block connector may also include a locking clip.

According to one aspect of the disclosure, an orthopaedic surgical instrument assembly for resecting a patient's tibia during an orthopaedic knee replacement procedure includes a tibial cutting block having a mediolaterally extending cutting slot and a tibial stylus instrument. The tibial stylus instrument includes a mounting flange configured to be positioned in the cutting slot so as to secure the tibial stylus instrument to the tibial cutting block. The tibial stylus instrument also includes a posteriorly extending medial stylus having a pointer configured to rest on a medial tibial plateau of the patient's tibia, and a medial position adjustment assembly operable to upwardly and downwardly adjust the position of the medial stylus relative to the mounting flange. The tibial stylus instrument further includes a posteriorly extending lateral stylus having a pointer configured to rest on a lateral tibial plateau of the patient's tibia, and a lateral position adjustment assembly operable to upwardly and downwardly adjust the position of the lateral stylus relative to the mounting flange.

In an example, the medial position adjustment assembly includes a housing coupled to a slider. The slider is upwardly and downwardly movable relative to the mounting flange, and the medial stylus is secured to the slider to move therewith.

In another example, the medial position adjustment assembly further includes a control knob. Rotation of the control knob in a first direction moves the slider upwardly relative to the mounting flange, whereas rotation of the control knob in a second, opposite direction moves the slider downwardly relative to the mounting flange.

In an example, the housing is coupled with the slider to move therewith,

the medial stylus extends through the housing, the medial stylus is moveable anteroposteriorly relative to the housing and the slider, and the medial stylus is moveable mediolaterally relative to the slider.

In an example, the lateral position adjustment assembly includes a housing coupled to a slider. The slider is upwardly and downwardly movable relative to the mounting flange, and the lateral stylus is secured to the slider to move therewith.

In another example, the lateral position adjustment assembly further includes a control knob. Rotation of the control knob in a first direction moves the slider upwardly relative to the mounting flange, whereas rotation of the control knob in a second, opposite direction moves the slider downwardly relative to the mounting flange.

In an example, the housing is coupled with the slider to move therewith,

the lateral stylus extends through the housing, the lateral stylus is moveable anteroposteriorly relative to the housing and the slider, and the lateral stylus is moveable mediolaterally relative to the slider.

In an example, movement of the medial and lateral styluses via operation of the medial and lateral position adjustment assemblies adjusts a cutting position of the tibial cutting block.

In an example, movement of the medial and lateral styluses via operation of the medial and lateral position adjustment assemblies adjusts a cutting angle of the tibial cutting block.

According to another aspect, an orthopaedic surgical instrument assembly for resecting a patient's tibia during an orthopaedic knee replacement procedure comprising a tibial cutting block having a mediolaterally extending cutting slot and a tibial stylus instrument. The tibial stylus instrument has a mounting flange configured to be positioned in the cutting slot so as to secure the tibial stylus instrument to the tibial cutting block, a posteriorly extending stylus having a pointer configured to rest on a tibial plateau of the patient's tibia, and a position adjustment assembly operable to upwardly and downwardly adjust the position of the stylus relative to the mounting flange. The position adjustment assembly includes a control knob. Rotation of the control knob in a first direction moves the stylus upwardly relative to the mounting flange, whereas rotation of the control knob in a second, opposite direction moves the stylus downwardly relative to the mounting flange.

In an example, the position adjustment assembly includes a housing coupled to a slider, the slider is upwardly and downwardly movable relative to the mounting flange, and the stylus is secured to the slider to move therewith.

In another example, the stylus extends through the housing, the stylus is moveable anteroposteriorly relative to the housing and the slider, and the stylus is moveable mediolaterally relative to the slider.

In an example, the stylus is a first stylus, and the position adjustment assembly is a first position adjustment assembly. The orthopaedic surgical instrument assembly further includes a second posteriorly extending stylus having a pointer configured to rest on another tibial plateau of the patient's tibia, and a second position adjustment assembly operable to upwardly and downwardly adjust the position of the second stylus relative to the mounting flange.

According to another aspect, a method of surgically preparing a patient's tibia during an orthopaedic surgical knee procedure includes assembling a tibial cutting block to a tibial stylus instrument, with the tibial cutting block having a mediolaterally extending cutting slot. A medial position adjustment assembly of the tibial stylus instrument is operated so as to position a medial stylus of the tibial stylus instrument a desired distance away from the cutting slot. A lateral position adjustment assembly of the tibial stylus instrument is operated so as to position a lateral stylus of the tibial stylus instrument a desired distance away from the cutting slot. The assembled tibial cutting block and tibial stylus instrument is positioned on the patient's tibia such that (i) a bone facing surface of the tibial cutting block abuts an anterior surface of the patient's tibia, (ii) a pointer of the medial stylus contacts a medial tibial plateau of the patient's tibia, and (iii) a pointer of the lateral stylus contacts a lateral tibial plateau of the patient's tibia.

In an example, the method further includes operating, with the pointer of the medial stylus in contact with the medial tibial plateau of the patient's tibia, the medial position adjustment assembly so as to adjust the position of the cutting slot relative to the patient's tibia.

In an example, the method further includes operating, with the pointer of the lateral stylus in contact with the lateral tibial plateau of the patient's tibia, the lateral position adjustment assembly so as to adjust the position of the cutting slot relative to the patient's tibia.

In an example, the method further includes operating, with (i) the pointer of the medial stylus in contact with the medial tibial plateau of the patient's tibia, and (ii) the pointer of the lateral stylus in contact with the lateral tibial plateau of the patient's tibia, one or both of the medial position adjustment assembly and the lateral position adjustment assembly so as to alter the angle of the cutting slot relative to the anterior surface of the patient's tibia.

Adjusting the angle of the cutting slot relative to the anterior surface of the patient's tibia may include adjusting a varus/valgus angle of the cutting slot.

A medial control knob may be rotated so as to position the medial stylus of the tibial stylus instrument the desired distance away from the cutting slot, and a lateral control knob may be rotated so as to position the lateral stylus of the tibial stylus instrument the desired distance away from the cutting slot.

According to another aspect, a method of surgically preparing a patient's tibia during an orthopaedic surgical knee procedure assembling a tibial cutting block to a tibial stylus instrument, with the tibial cutting block having a mediolaterally extending cutting slot. The assembled tibial cutting block and tibial stylus instrument are positioned on the patient's tibia such that (i) a bone facing surface of the tibial cutting block abuts an anterior surface of the patient's tibia, (ii) a pointer of the medial stylus contacts a medial tibial plateau of the patient's tibia, and (iii) a pointer of the lateral stylus contacts a lateral tibial plateau of the patient's tibia. With the pointer of the medial stylus in contact with the medial tibial plateau of the patient's tibia, a medial position adjustment assembly of the tibial stylus instrument is operated so as to adjust the position of the cutting slot relative to the patient's tibia. With the pointer of the lateral stylus in contact with the lateral tibial plateau of the patient's tibia, a lateral position adjustment assembly of the tibial stylus instrument is operated so as to adjust the position of the cutting slot relative to the patient's tibia.

In an example, one or both of the medial position adjustment assembly and the lateral position adjustment assembly is operated so as to alter the angle of the cutting slot relative to the anterior surface of the patient's tibia.

Adjusting the angle of the cutting slot relative to the anterior surface of the patient's tibia may include adjusting a varus/valgus angle of the cutting slot.

A medial control knob may be rotated so as to position the medial stylus of the tibial stylus instrument the desired distance away from the cutting slot, and a lateral control knob may be rotated so as to position the lateral stylus of the tibial stylus instrument the desired distance away from the cutting slot.

In an example, the method also includes determining an amount of cartilage loss on one or both of the medial and lateral tibial plateaus of the patient's tibia. The position of the cutting slot relative to the anterior surface of the patient's tibia may be altered based on the determined amount of cartilage loss on one or both of the medial and lateral tibial plateaus of the patient's tibia.

### BRIEF DESCRIPTION OF THE DRAWINGS

The detailed description particularly refers to the following figures, in which:

FIG. 1 is a perspective view of a tibial alignment jig for use in the surgical preparation of a patient's tibia during performance of an orthopaedic knee procedure;

FIG. 2 is an enlarged front view of the proximal tibial jig assembly of the tibial alignment jig of FIG. 1;

FIG. 3 is an enlarged side view of the proximal tibial jig assembly of the tibial alignment jig of FIG. 1;

FIG. 4 is a perspective view of a tibial stylus instrument for use in the surgical preparation of a patient's tibia during performance of an orthopaedic knee procedure;

FIG. 5 is a fragmentary side view of the tibial alignment jig of FIG. 1 installed on the patient's tibia, note that much of the distal tibial jig assembly has been cut away in FIG. 5 for clarity of description;

FIG. 6 is a fragmentary front view of the tibial alignment jig of FIG. 1 installed on the patient's tibia, note that much of the distal tibial jig assembly has been cut away in FIG. 6 for clarity of description;

FIG. 7 is a fragmentary perspective view showing the tibial alignment jig and the tibial stylus instrument installed on the proximal end of the patient's tibia prior to performance of a resection of the patient's tibia;

FIG. 8 is a top view showing the tibial stylus instrument installed on the proximal end of the patient's tibia prior to performance of a resection of the patient's tibia, note the tibial alignment jig is not shown in FIG. 8 for clarity of description;

FIG. 9 is a perspective view of another tibial alignment jig for use in the surgical preparation of a patient's tibia during performance of an orthopaedic knee procedure;

FIG. 10 is an enlarged perspective view of the proximal tibial jig assembly of the tibial alignment jig of FIG. 9;

FIG. 11 is a fragmentary cross sectional view taken along the line 11-11 of FIG. 10, as viewed in the direction of the arrows;

FIG. 12 is a fragmentary cross sectional view taken along the line 12-12 of FIG. 10, as viewed in the direction of the arrows;

FIG. 13 is a perspective view of another tibial stylus instrument for use in the surgical preparation of a patient's tibia during performance of an orthopaedic knee procedure;

FIG. 14 is a fragmentary side view of a portion of the tibial stylus instrument of FIG. 13; and

FIG. 15 is a fragmentary front view of a portion of the tibial stylus instrument of FIG. 13.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, proximal, distal, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

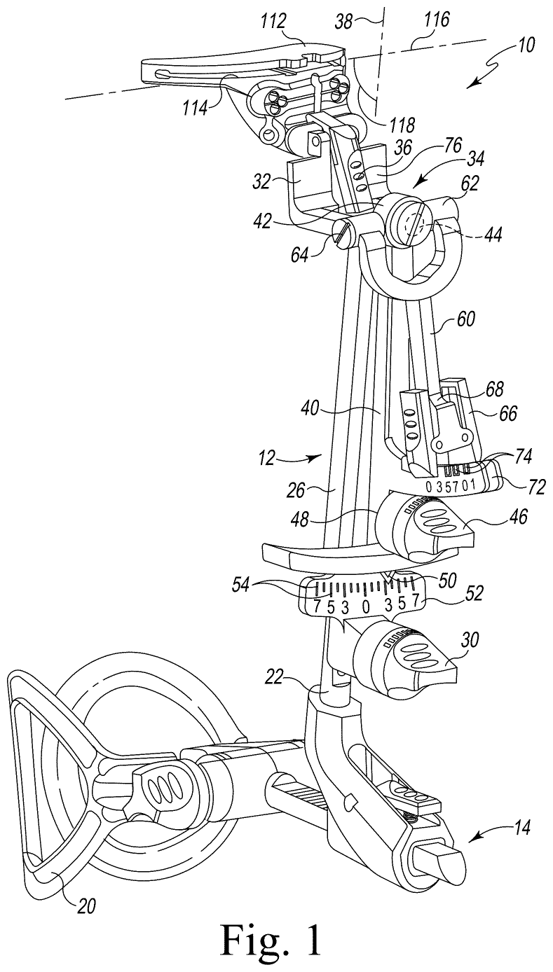

Referring to FIGS. 1-3, an orthopaedic surgical instrument 10 in the form of a tibial alignment jig-for use in the surgical preparation of a patient's tibia during performance of an orthopaedic knee procedure is shown. The tibial alignment jig 10 includes a proximal tibial jig assembly 12 and a distal tibial jig assembly 14. The tibial alignment jig 10 is used to position and install a tibial cutting block 112 (see FIGS. 1, 5-7) that is used to perform a proximal cut on the patient's tibia.

The tibial alignment jig 10 may be used both by surgeons with a preference for mechanical alignment techniques and those surgeons with a preference for kinematic alignment techniques. Broadly, mechanical alignment techniques involve determining the resection planes based on a pre-determined angle as a function of mechanical alignment, whereas kinematic alignment techniques involve determining the resection planes as a function of the native, pre-disease state of the patient's knee.

As can be seen in FIG. 1, the distal tibial jig assembly 14 includes an extramedullary ankle clamp 20 positioned on the assembly's inferior end. An alignment rod 22 extends superiorly away from the ankle clamp 20. As will be discussed in more detail below, the ankle clamp 20 is positioned around the malleoli of the patient's ankle such that the alignment rod 22 extends superiorly in alignment with the tibial axis of the patient's tibia 120.

As can be seen in FIGS. 1-3, the proximal tibial jig assembly 12 includes a hollow extramedullary proximal rod 26. The proximal rod 26 has an elongated bore 28 formed therein that is configured to receive the alignment rod 22 of the distal tibial jig assembly 14. A locking knob 30 is used to lock the position of the alignment rod 22 (and hence the distal tibial jig assembly 14) relative to the proximal rod 26 (and hence the proximal tibial jig assembly 12). The locking knob 30 has an internal shaft that extends inwardly into the bore 28 of the proximal rod 26 and is displaced by rotation of the locking knob 30 to selectively clamp the alignment rod 22 of the distal tibial jig assembly 14 in a locked position within the proximal rod 26. In particular, when the locking knob 30 is loosened, the two rods 22, 26 are free to slide relative to one another so as to adjust the overall length of the tibial alignment jig 10. Once a desired length is achieved, the locking knob 30 may be tightened to lock the position of the two rods 22, 26 relative to one another. It should be appreciated that such changing of the length of the assembled tibial alignment jig 10 adjusts the superoinferior position of the cutting block 112 on the anterior surface 128 of the patient's tibia 120 thereby determining the resection height at which the block's cutting slot 114 is positioned.

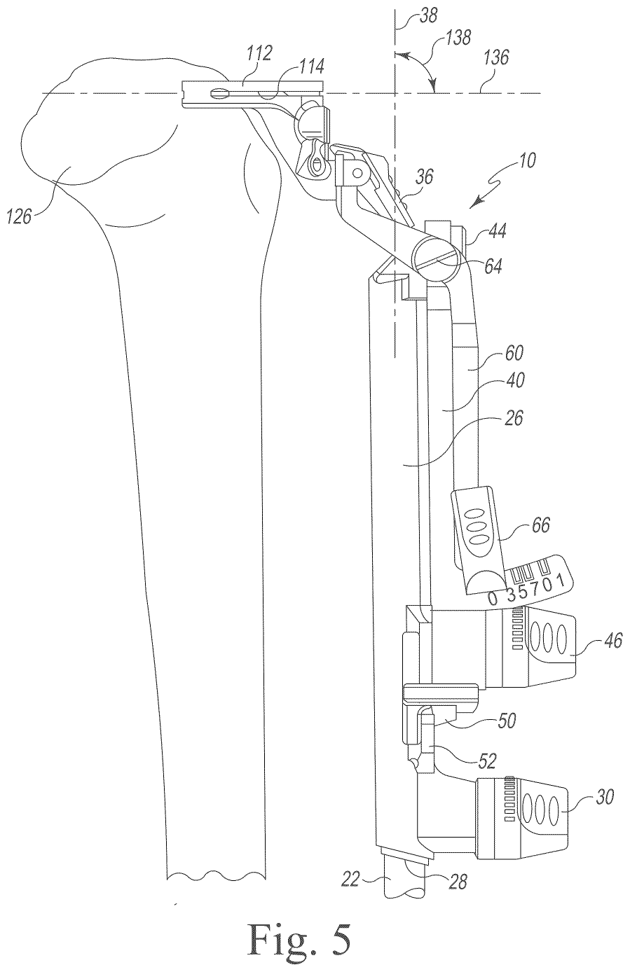

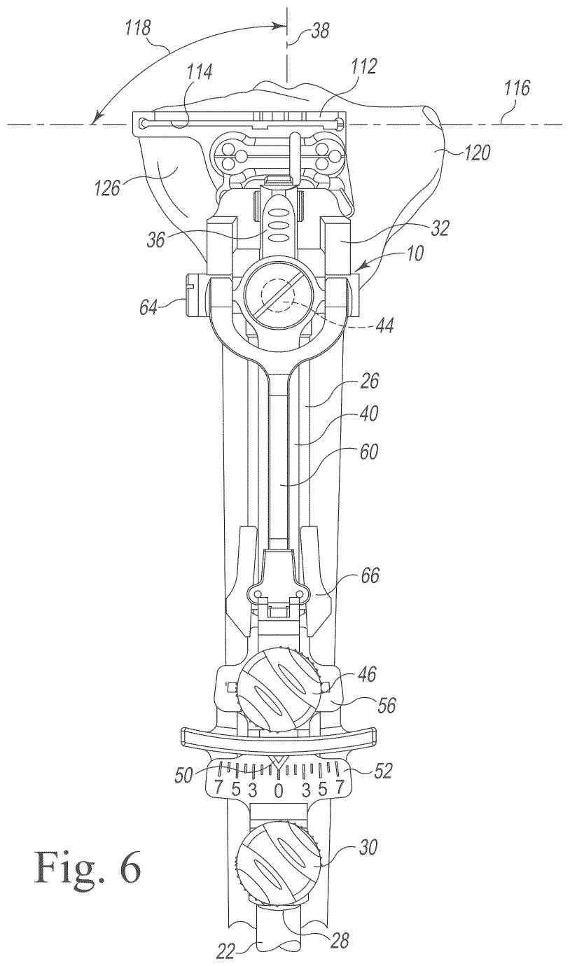

A block connector 32 is positioned on a superior end 34 of the proximal tibial jig assembly 12. In the illustrative embodiment described herein, the block connector 32 includes a mounting clip 36 and a pair of mounting pins 78, although other types of connectors are contemplated for use. The tibial cutting block 112 is clipped or otherwise secured to the proximal tibial jig assembly 12 by advancing the mounting pins 78 into a pair of corresponding holes in the cutting block 112 and then retaining the cutting block 112 against a clip body 76 with the mounting clip 36. As can be seen in FIGS. 1 and 6, when the tibial cutting block 112 is secured to the proximal tibial jig assembly 12, the common longitudinal axis 38 of the rods 22, 26 and the mediolaterally-extending longitudinal axis 116 of the cutting slot 114 of the tibial cutting block 112 define a varus/valgus cutting angle 118 (see FIG. 6). As will be discussed below in more detail, as the tibial cutting block 112 is rotated relative to the proximal rod 26 about an anteroposteriorly extending axis, the orientation of the mediolaterally-extending longitudinal axis 116 of the cutting slot 114 of the tibial cutting block 112 relative to the longitudinal axis 38 of the rods 22, 26 likewise is rotated thereby changing the varus/valgus cutting angle 118. Moreover, the common longitudinal axis 38 of the rods 22, 26 and the anteroposteriorly-extending cutting plane 136 defined by the guide surface of the cutting slot 114 of the tibial cutting block 112 defines a posterior slope cutting angle 138 (see FIG. 5). As will be discussed below in more detail, as the tibial cutting block 112 is rotated relative to the proximal rod 26 about a mediolaterally-extending axis, the orientation of the cutting plane 136 of the cutting slot 114 of the tibial cutting block 112 relative to the longitudinal axis 38 of the rods 22, 26 likewise is rotated thereby changing the posterior slope cutting angle 138.

As can be seen in FIGS. 1-3, the proximal tibial jig assembly 12 includes a varus/valgus adjustment arm 40. A superior end 42 of the varus/valgus adjustment arm 40 is coupled to the block connector 32 and pivotally coupled to the proximal rod 26 via an anteroposteriorly extending pivot joint defined by an anteroposteriorly extending pivot pin 44. Rotation of the varus/valgus adjustment arm 40 about the pivot pin 44 adjusts the varus/valgus cutting angle 118 of the cutting slot 114 of the tibial cutting block 112. A locking knob 46 is secured to an inferior end 48 of the varus/valgus adjustment arm 40. The locking knob 46 is operable to lock the rotational position of the varus/valgus adjustment arm 40 (and hence the tibial cutting block 112 secured to the block connector 32) relative to the proximal rod 26. The locking knob 46 has an internal shaft that extends inwardly toward a locking plate 56 of the proximal rod 26 and is displaced by rotation of the locking knob 46 to selectively clamp the varus/valgus adjustment arm 40 in a locked position relative to the proximal rod 26. In particular, when the locking knob 46 is loosened, the varus/valgus adjustment arm 40 is free to rotate relative to the proximal rod 26 about the anteroposteriorly extending pivot pin 44. Once a desired varus/valgus cutting angle 118 of the cutting slot 114 of the tibial cutting block 112 is achieved, the locking knob 46 may be tightened to lock the position of the varus/valgus adjustment arm 40 (and hence the tibial cutting block 112 secured to the block connector 32) relative to the proximal rod 26.

As can be seen inf FIGS. 1-3, a position indicator in the form of a pointer 50 extends inferiorly from the inferior end 48 of the varus/valgus adjustment arm 40. An indexing plate 52 is secured to the proximal rod 26 at a location near the locking knob 30. The pointer 50 moves radially along the indexing plate 52 during rotation of the varus/valgus adjustment arm 40 relative to proximal rod 26 about the anteroposteriorly extending pivot pin 44. The indexing plate 52 has indicia in the form of a plurality of angle indicators 54 radially engraved, printed, or otherwise formed thereon. Each of the plurality of angle indicators 54 represents a desired varus/valgus angle (e.g., left or right −0 degrees to 7 degrees) for selection by a surgeon during use of the tibial alignment jig 10. As will be described in more detail below, such use of the locking knob 46 and the indexing plate 52 allows the tibial alignment jig 10 to be operated in either a mechanical alignment mode of operation or a kinematic alignment mode of operation.

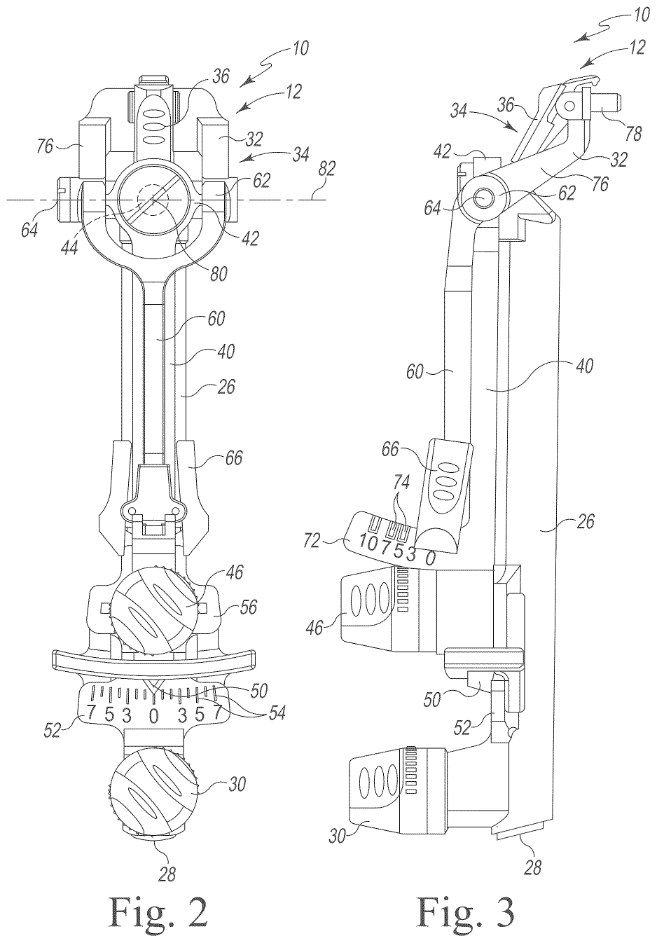

As can be seen in FIGS. 1-3, the proximal tibial jig assembly 12 includes a posterior slope adjustment arm 60. A superior end 62 of the posterior slope adjustment arm 60 is pivotally coupled to the varus/valgus adjustment arm 40 via a mediolaterally extending pivot joint defined by a mediolaterally extending pivot pin 64. Rotation of the posterior slope adjustment arm 60 about the pivot pin 64 adjusts the posterior slope angle of the cutting slot 114 of the tibial cutting block 112. Moreover, as shown in FIG. 1, by virtue of being coupled to the superior end 42 of the varus/valgus adjustment arm 40, the posterior slope adjustment arm 60 rotates with the varus/valgus adjustment arm 40 about the anteroposteriorly extending pivot pin 44. In other words, the posterior slope adjustment arm 60 rotates relative to the proximal rod 26 about both the anteroposteriorly extending pivot pin 44 and the mediolaterally extending pivot pin 64. As can be seen in FIG. 2, in the example described herein, the rotational axis 80 of the pivot joint defined by the anteroposteriorly extending pivot pin 44 and the rotational axis 82 of the pivot joint defined by the mediolaterally extending pivot pin 64 are coplanar and thus intersect one another at an orthogonal angle.

The superior end 62 of the posterior slope adjustment arm 60 is coupled to the block connector 32. In the illustrative example disclosed herein, the clip body 76 of the block connector 32 is integrally formed in the superior end 62 of the posterior slope adjustment arm 60. In such a way, the block connector 32 rotates relative to the proximal rod 26 about both the anteroposteriorly extending pivot pin 44 and the mediolaterally extending pivot pin 64.

A locking lever 66 is secured to an inferior end 68 of the posterior slope adjustment arm 60. The locking lever 66 is operable to lock the rotational position of the posterior slope adjustment arm 60 (and hence the tibial cutting block 112 secured to the block connector 32) relative to the varus/valgus adjustment arm 40. In particular, an indexing plate 72 extends outwardly from the varus/valgus adjustment arm 40 at a location near the locking knob 46. The indexing plate 72 has a plurality of locking notches 74 radially positioned along its length. Each of the plurality of locking notches 74 represents a desired posterior slope angle (e.g., 0 degrees to 10 degrees) for selection by a surgeon during use of the tibial alignment jig 10. The locking lever 66 is spring-loaded or otherwise urged in the direction of the locking notches 74. Thus, when the locking lever 66 is squeezed or otherwise disengaged from the locking notches 74, the posterior slope adjustment arm 60 is free to rotate relative to the varus/valgus adjustment arm 40 (and hence the proximal rod 26) about the mediolaterally extending pivot pin 64. Once a desired posterior slope cutting angle 138 of the cutting slot 114 of the tibial cutting block 112 is achieved, the locking lever 66 may be released or otherwise reengaged with the corresponding locking notch 74 to lock the position of the posterior slope adjustment arm 60 (and hence the tibial cutting block 112 secured to the block connector 32) relative to the varus/valgus adjustment arm 40 (and hence the proximal rod 26).

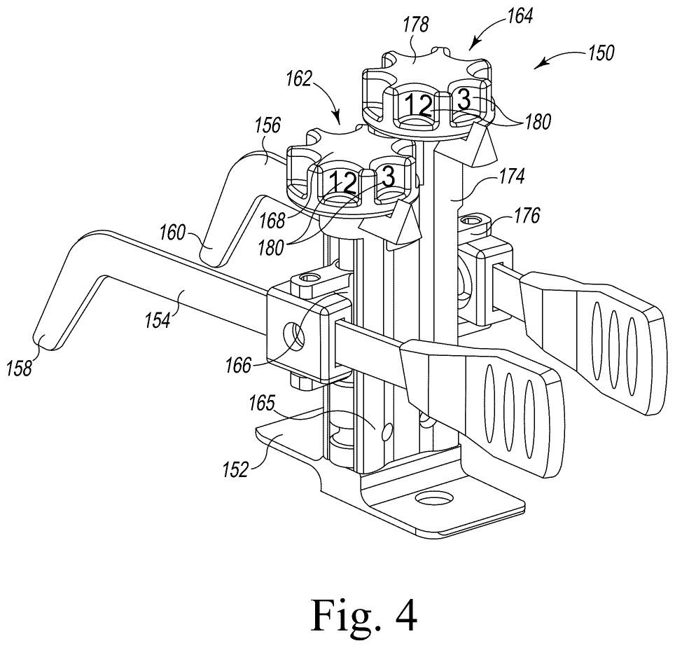

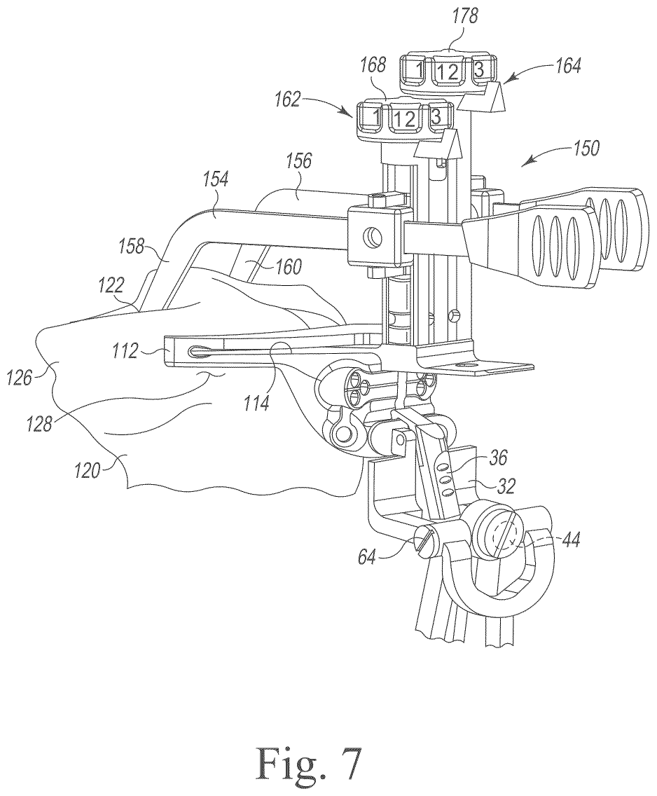

As described above, the surgeon may use the tibial alignment jig 10 to adjust the position of the cutting slot 114 of the tibial cutting block 112 relative to the patient's tibia 120 so as to adjust the block's (1) superoinferior position or “height” of the cutting slot 114, (2) the varus/valgus cutting angle 118 of the cutting slot 114 (and hence the varus/valgus angle in which the tibial prosthetic component is implanted), and (3) the posterior slope cutting angle 138 of the cutting slot 114 (and hence the posterior slope angle in which the tibial prosthetic component is implanted). As will be discussed below in more detail, the tibial alignment jig 10 may be used in conjunction with a tibial stylus instrument 150 (see FIGS. 4 and 7) during such positioning of the tibial cutting block 112. The tibial alignment jig 10 may be operated in two different modes of operation based on a given surgeon's preferences. In particular, for a surgeon with a preference for mechanical alignment techniques, the tibial alignment jig 10 may be operated in a mechanical alignment mode of operation. Typically, a surgeon utilizing mechanical alignment techniques will prefer to (1) set the height of the cutting block 112 based on the thickness of a preoperatively selected tibial implant (e.g., 9 mm thick implant), (2) set the varus/valgus cutting angle 118 at a predetermined angle (e.g., 5-7 degrees) relative to the anatomic axis of the tibia 120 based on preoperative planning, and (3) set the posterior slope cutting angle 138 based on the type of tibial implant being implanted (e.g., 3 degrees if a posterior stabilized knee prosthetic is being implanted or 5-7 degrees if a cruciate retaining prosthetic is being implanted). Thus, the surgeon may operate the tibial alignment jig 10 in the mechanical alignment mode of operation by (1) extending the alignment rod 22 (and hence the distal tibial jig assembly 14) relative to the proximal rod 26 (and hence the proximal tibial jig assembly 12) into a length that positions the cutting block 112 at the desired, preoperatively-determined height and tightening the locking knob 30 to lock the position of the two rods 22, 26 relative to one another, (2) positioning the varus/valgus adjustment arm 40 in a rotational position corresponding to the desired, preoperatively determined varus/valgus cutting angle 118 (e.g., left or right −0 degrees to 7 degrees) and tightening the locking knob 46 so as to lock the rotational position of the varus/valgus adjustment arm 40 relative to the proximal rod 26 in a position that positions the cutting slot 114 of the tibial cutting block 112 in the desired varus/valgus cutting angle 118 (e.g., left or right −0 degrees to 7 degrees), and (3) positioning the posterior slope adjustment arm 60 in a rotational position corresponding to the desired, preoperatively determined posterior slope cutting angle 138 (e.g., 0 degrees to 10 degrees) and locking the locking lever 66 so as to lock the rotational position of the posterior slope adjustment arm 60 relative to the varus/valgus adjustment arm 40 (and hence the proximal rod 26) in a position that positions the cutting slot 114 of the tibial cutting block 112 in the desired posterior slope cutting angle 138 (e.g., 0 degrees to 10 degrees).

Alternatively, for a surgeon with a preference for kinematic alignment techniques, the tibial alignment jig 10 may be operated in a kinematic alignment mode of operation. Typically, a surgeon utilizing kinematic alignment techniques will prefer to intraoperatively set the height and varus/valgus cutting angle 118 of the cutting block 112. To make such intraoperative determinations, the surgeon typically sets the posterior slope cutting angle 138 to neutral (i.e., 0 degrees) and then “floats” the height and varus/valgus cutting angle 118 of the tibial cutting block 112, as opposed to selecting from specific predetermined positions as is the case with mechanical alignment techniques. As a result, the surgeon may operate the tibial alignment jig 10 in the kinematic mode of operation by (1) positioning the posterior slope adjustment arm 60 in a rotational position corresponding to a neutral posterior slope cutting angle 138 (i.e., 0 degrees) and locking the locking lever 66 so as to lock the rotational position of the posterior slope adjustment arm 60 relative to the varus/valgus adjustment arm 40 (and hence the proximal rod 26) in a position that positions the cutting slot 114 of the tibial cutting block 112 in the neutral posterior slope cutting angle 138 (i.e., 0 degrees), (2) loosening the locking knob 30 so as to allow the alignment rod 22 and the proximal rod 26 to slide freely relative to one another (e.g., “float”) to allow the surgeon to position the cutting slot 114 of the tibial cutting block 112 in a desired superoinferior position (i.e., height) relative to the patient's tibia 120 and thereafter tightening the locking knob 30 so as to lock the rods 22, 26 relative to one another in a position that positions the cutting slot 114 of the tibial cutting block 112 in the desired intraoperatively-determined height, and (3) loosening the locking knob 46 so as to allow the varus/valgus adjustment arm 40 to rotate freely (e.g., “float”) relative to the proximal rod 26 to allow the surgeon to position the cutting block 112 in a desired varus/valgus cutting angle 118 and thereafter tightening the locking knob 46 so as to lock the rotational position of the varus/valgus adjustment arm 40 relative to the proximal rod 26 in a position that positions the cutting slot 114 of the tibial cutting block 112 in the desired intraoperatively-determined varus/valgus cutting angle 118.

Referring now to FIG. 4, there is shown the tibial stylus instrument 150 in more detail. As will be described in more detail below, in use, the tibial stylus instrument 150 contacts the tibial plateaus 122, 124 of the patient's tibia 120 to facilitate positioning of the tibial cutting block 112. As will be described below in more detail, like the tibial alignment jig 10, the tibial stylus instrument 150 may be used both by surgeons with a preference for mechanical alignment techniques and those surgeons having a preference for kinematic alignment techniques.

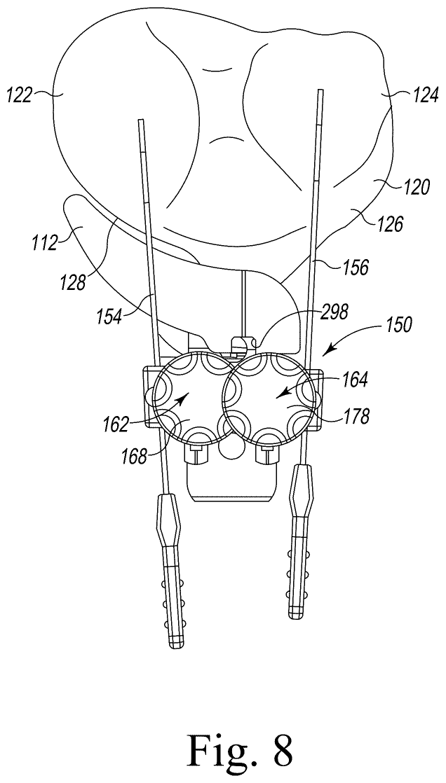

The tibial stylus instrument 150 includes a mounting flange 152 configured to be positioned in the cutting slot 114 of the tibial cutting block 112 so as to secure the tibial stylus instrument 150 to the cutting block 112. The tibial stylus instrument 150 also includes a pair of posteriorly extending styluses 154, 156 that are configured to contact the tibial plateaus 122, 124 of the patient's tibia 120. In particular, as shown best in FIGS. 4 and 8, the medial stylus 154 has a pointer 158 formed in its posterior end that is configured to rest on the medial tibial plateau 122 of the patient's tibia 120. Similarly, the lateral stylus 156 has a pointer 160 formed in its posterior end that is configured to rest on the lateral tibial plateau 124 of the patient's tibia 120.

The tibial stylus instrument 150 also includes a pair of position adjustment assemblies 162, 164 that are independently operable to upwardly and downwardly (i.e., superiorly and inferiorly) adjust the position of the styluses 154, 156 relative to the mounting flange 152 (and hence the cutting slot 114 when the instrument 150 is coupled to the cutting block 112), respectively. As can be seen in FIGS. 4 and 7, the medial position adjustment assembly 162 includes a housing 165 having a slider 166 positioned therein. The medial stylus 154 is secured to the slider 166. A control knob 168 is operable to move the slider 166 within the housing 165 to adjust the position of the medial stylus 154 relative to the mounting flange 152 (and hence the cutting slot 114 when the instrument 150 is coupled to the cutting block 112). In particular, rotation of the control knob 168 in a first direction (e.g., clockwise) moves the slider 166 upwardly in the housing 165 thereby moving the medial stylus 154 upwardly (i.e., superiorly) in the direction away from the mounting flange 152 (and hence the cutting slot 114 when the instrument 150 is coupled to the cutting block 112). Conversely, rotation of the control knob 168 in a second, opposite direction (e.g., counterclockwise) moves the slider 166 downwardly in the housing 165 thereby moving the medial stylus 154 downwardly (i.e., inferiorly) in the direction toward the mounting flange 152 (and hence the cutting slot 114 when the instrument 150 is coupled to the cutting block 112).

Similarly, the lateral position adjustment assembly 164 includes a housing 174 having a slider 176 positioned therein. The lateral stylus 156 is secured to the slider 176. A control knob 178 is operable to move the slider 176 within the housing 174 to adjust the position of the lateral stylus 156 relative to the mounting flange 152 (and hence the cutting slot 114 when the instrument 150 is coupled to the cutting block 112). In particular, rotation of the control knob 178 in a first direction (e.g., clockwise) moves the slider 176 upwardly in the housing 174 thereby moving the lateral stylus 156 upwardly (i.e., superiorly) in the direction away from the mounting flange 152 (and hence the cutting slot 114 when the instrument 150 is coupled to the cutting block 112). Conversely, rotation of the control knob 178 in a second, opposite direction (e.g., counterclockwise) moves the slider 176 downwardly in the housing 174 thereby moving the lateral stylus 156 downwardly (i.e., inferiorly) in the direction toward the mounting flange 152 (and hence the cutting slot 114 when the instrument 150 is coupled to the cutting block 112).

As can be seen in FIGS. 4 and 7, the control knobs 168, 178 have indicia in the form of a plurality of resection level indicators 180 engraved, printed, or otherwise formed thereon. Each of the plurality of resection level indicators 180 corresponds to a resection amount, as measured in millimeters (e.g., 3 mm to 13 mm) for selection by a surgeon during use of the tibial stylus instrument 150. A surgeon may operate the position adjustment assemblies 162, 164 to change the height of the cutting slot 114 when the tibial stylus instrument 150 is coupled to the tibial cutting block 112. In particular, the surgeon may rotate one or both the control knobs 168, 178 to a desired resection level indicator 180 to adjust upwardly or downwardly the height of the block's cutting slot 114 on the patient's tibia 120. While doing so, the surgeon may also adjust the cutting angle of the block's cutting slot 114 by rotating the control knobs 168, 178 to different resection level indicators 180.

As alluded to above, the tibial stylus instrument 150 is operable for use both by surgeons with a preference for mechanical alignment techniques and those surgeons having a preference for kinematic alignment techniques. In the case of a mechanical alignment technique, the surgeon generally determines preoperatively the desired resection height based on the thickness of the planned tibial prosthetic to be implanted. For example, a common resection height is 9 mm based on an implant construct that includes a 4 mm thick tibial tray and a 5 mm thick tibial bearing. In such an example, the surgeon would generally use only one of the styluses−typically the medial stylus 154−to position the cutting block 112 at the resection level. To do so, the surgeon would rotate the control knob 168 of the medial position adjustment assembly 162 to the “9” resection level indicator 180. Thereafter, the surgeon would secure the tibial stylus instrument 150 to the tibial cutting block 112 (which itself may already be clipped to the tibial alignment jig 10) by inserting the instrument's mounting flange 152 into the block's cutting slot 114 as shown in FIG. 7. The height of the tibial alignment jig 10 is then adjusted until the pointer 158 formed in the posterior end of the medial stylus 154 rests on the lowest point (i.e., the dwell point) of the medial tibial plateau 122 of the patient's tibia 120.

In the case of a kinematic alignment technique, the surgeon generally follows a similar approach, but instead uses both styluses 154, 156 to position the tibial cutting block 112. To do so, in the example of a desired 9 mm resection level, the surgeon would rotate both the control knobs 168, 178 of the medial and lateral position adjustment assemblies 162, 164, respectively, to the “9” resection level indicator 180. Thereafter, the surgeon would secure the tibial stylus instrument 150 to the tibial cutting block 112 (which itself may already be clipped to the tibial alignment jig 10) by inserting the instrument's mounting flange 152 into the block's cutting slot 114 as shown in FIG. 7. The height of the tibial alignment jig 10 is then adjusted until the pointer 158 formed in the posterior end of the medial stylus 154 rests on the lowest point (i.e., the dwell point) of the medial tibial plateau 122 of the patient's tibia 120 and the pointer 160 formed in the posterior end of the lateral stylus 156 rests on the lowest point (i.e., the dwell point) of the lateral tibial plateau 124 of the patient's tibia 120. If need be, the surgeon may change the position of the varus/valgus adjustment arm 40 to position the tibial stylus instrument 150 in a position in which both pointers 158, 160 are positioned on the dwell points of the respective tibial plateaus 122, 124.

During such use in a kinematic alignment technique, the surgeon may also intraoperatively adjust the settings of the tibial stylus instrument 150 to intraoperatively adjust the resection level based on intraoperative observations or measurements taken by the surgeon. For example, the surgeon may intraoperatively determine or otherwise estimate an amount of cartilage loss on one or both of the tibial plateaus 122, 124 of the patient's tibia 120. The surgeon may then utilize the tibial stylus instrument 150 to accommodate for such cartilage loss by adjusting the resection level of the side or sides of the patient's tibia 120 experiencing cartilage loss. For instance, if a surgeon preoperatively planned a 9 mm resection level (based on the planned use of a tibial construct having a 4 mm thick tibial tray and a 5 mm thick tibial bearing), but then intraoperatively determined that, for example, both of the patient's tibial plateaus 122, 124 exhibited 2 mm of cartilage loss, the surgeon may set the control knobs 168, 178 of the medial and lateral position adjustment assemblies 162, 164, respectively, to the “7” resection level indicator 180 to reduce the resection level by the measured amount of cartilage loss (e.g., 2 mm in the example described herein). Thereafter, the surgeon would use the tibial stylus instrument 150 to position the tibial cutting block 112 in the manner described above. By reducing the resection level to accommodate for the measured amount of cartilage loss, the tibial prosthetic may be implanted at a height which mimics the patient's native anatomy to what it was prior to the onset of cartilage loss (i.e., prior to the onset of disease or trauma which reduced the patient's natural cartilage).

In operation, the surgeon may utilize the tibial alignment jig 10 and the tibial stylus instrument 150 during performance of an orthopaedic knee procedure to prepare the proximal end 126 of the patient's tibia 120 to receive a prosthetic tibial component. To do so, the surgeon may utilize the tibial alignment jig 10 and the tibial stylus instrument 150 to position and install (e.g., pin) the tibial cutting block 112 on the patient's tibia 120 and thereafter use the installed cutting block 112 to guide a bone saw blade in making a tibial cut on the proximal end 126 of the patient's tibia 120.

During such an orthopaedic surgical procedure, the surgeon first orientates the patient's tibia 120 such that the patient's knee is positioned in flexion. With the patient's knee positioned in flexion, the surgeon assembles a surgical instrument construct. Specifically, the surgeon advances the alignment rod 22 of the distal tibial jig assembly 14 into the elongated bore 28 of the extramedullary proximal rod 26 of the proximal tibial jig assembly 12. The surgeon positions the two rods 22, 26 so as to produce a construct with an initial, preplanned length and then uses the locking knob 30 to lock the position of the alignment rod 22 (and hence the distal tibial jig assembly 14) relative to the proximal rod 26 (and hence the proximal tibial jig assembly 12).

Thereafter, the surgeon positions the ankle clamp 20 of the distal tibial jig assembly 14 around the malleoli of the patient's ankle such that the rods 22, 26 extend superiorly in alignment with the tibial axis of the patient's tibia 120 (see FIGS. 5 and 6). Doing so positions the tibial cutting block 112 such that its bone facing surface abuts the anterior surface 128 of the patient's tibia 120. The surgeon then secures the tibial stylus instrument 150 to the tibial cutting block 112 by inserting the instrument's mounting flange 152 into the block's cutting slot 114 as shown in FIGS. 7 and 8.

Once the tibial alignment jig 10 and the tibial stylus instrument 150 have been coupled to the patient's tibia 120, the surgeon uses the tibial alignment jig 10 and the tibial stylus instrument 150 to adjust the position of the cutting slot 114 of the tibial cutting block 112 relative to the patient's tibia 120 so as to adjust one or more of (1) the resection height of the cutting block 112, (2) the block's varus/valgus cutting angle 118, and/or (3) the posterior slope cutting angle 138 of the cutting block 112. As noted above, both the tibial alignment jig 10 and the tibial stylus instrument 150 may be operated in two different modes of operation based on the surgeon's preferences. If the surgeon has a preference for mechanical alignment techniques, the surgeon will generally (1) set the resection height of the cutting block 112 based on the thickness of a preoperatively selected tibial implant (e.g., 9 mm thick implant), (2) set the varus/valgus cutting angle 118 at a predetermined angle (e.g., 5-7 degrees) relative to the anatomic axis of the tibia 120 based on preoperative planning, and (3) set the posterior slope cutting angle 138 based on the type of prosthetic being implanted (e.g., 3 degrees if a posterior stabilized knee prosthetic is being implanted or 5-7 degrees if a cruciate retaining prosthetic is being implanted). To do so, the surgeon would rotate the control knob 168 of the medial position adjustment assembly 162 of the tibial stylus instrument 150 to the desired resection level indicator 180 (e.g., the “9” resection level indicator 180 in the case of a 9 mm resection level). Thereafter, the surgeon would then lower the pointer 158 formed in the posterior end of the medial stylus 154 until it rests on the lowest point (i.e., the dwell point) of the medial tibial plateau 122 of the patient's tibia 120 thereby positioning the cutting block 112 at the desired, preoperatively-determined height. The surgeon then tightens the locking knob 30 to lock the position of the two rods 22, 26 relative to one another.

If not already done, the surgeon then positions the varus/valgus adjustment arm 40 in a rotational position corresponding to the desired, preoperatively determined varus/valgus cutting angle 118 (e.g., left or right −0 degrees to 7 degrees) and tightens the locking knob 46 so as to lock the rotational position of the varus/valgus adjustment arm 40 relative to the proximal rod 26 in a position that positions the cutting slot 114 of the tibial cutting block 112 in the desired varus/valgus cutting angle 118 (e.g., left or right −0 degrees to 7 degrees). Likewise, if not already done, the surgeon positions the posterior slope adjustment arm 60 in a rotational position corresponding to the desired, preoperatively-determined posterior slope cutting angle 138 (e.g., 0-10 degrees) and locks the locking lever 66 so as to lock the rotational position of the posterior slope adjustment arm 60 relative to the varus/valgus adjustment arm 40 (and hence the proximal rod 26) in a position that positions the cutting slot 114 of the tibial cutting block 112 in the desired posterior slope cutting angle 138 (e.g., 0 degrees to 10 degrees).

Alternatively, if the surgeon has a preference for kinematic alignment techniques, the surgeon operates the tibial alignment jig 10 and the tibial stylus instrument 150 in a kinematic alignment mode of operation. In such a case, the surgeon intraoperatively sets one or more of (1) the resection height of the cutting block 112, (2) the block's varus/valgus cutting angle 118, and/or (3) the posterior slope cutting angle 138 of the cutting block 112. To do this, the surgeon would first rotate both the control knobs 168, 178 of the stylus instrument's medial and lateral position adjustment assemblies 162, 164, respectively, to the resection level indicator 180 corresponding to the desired resection height (e.g., in the example of a desired 9 mm resection level, the “9” resection level indicator 180). Thereafter, the surgeon lowers the height of the tibial alignment jig 10 until the pointer 158 formed in the posterior end of the medial stylus 154 rests on the lowest point (i.e., the dwell point) of the medial tibial plateau 122 of the patient's tibia 120 and the pointer 160 formed in the posterior end of the lateral stylus 156 rests on the lowest point (i.e., the dwell point) of the lateral tibial plateau 124 of the patient's tibia 120. While doing so, the surgeon leaves both the locking knobs 30, 46 loosened so as to “float” both the height and the varus/valgus angle adjustments−as opposed to the use of preoperatively-determined specific settings. As such, the surgeon may dynamically change both the length of the tibial alignment jig 10 (and hence the height of the cutting block 112) and the position of the varus/valgus adjustment arm 40 so as to position the tibial stylus instrument 150 in a position in which both pointers 158, 160 are positioned on the dwell points of the respective tibial plateaus 122, 124. Once satisfied with the alignment of the styluses 154, 156, the surgeon then tightens the locking knob 30 to lock the position of the two rods 22, 26 relative to one another and hence lock the resection level of the cutting block 112. The surgeon also tightens the locking knob 46 so as to lock the rotational position of the varus/valgus adjustment arm 40 relative to the proximal rod 26 in a position that positions the cutting slot 114 of the tibial cutting block 112 in the desired varus/valgus cutting angle 118. Typically, to facilitate making such intraoperative determinations, the surgeon sets the posterior slope cutting angle 138 to neutral (i.e., 0 degrees).

During such use in a kinematic alignment technique, the surgeon may also intraoperatively adjust the settings of the tibial stylus instrument 150 to intraoperatively adjust the resection level based on intraoperative observations or measurements taken by the surgeon. For example, as noted above, the surgeon may intraoperatively determine or otherwise estimate an amount of cartilage loss on one or both of the tibial plateaus 122, 124 of the patient's tibia 120. To do so, the surgeon determines the amount of cartilage loss on an affected area of the medial tibial plateau 122 (i.e., an area of the plateau 122 exhibiting cartilage loss) by inserting a graduated depth probe (not shown) or similar instrument into the affected area of the medial tibial plateau 122 and then also inserting the graduated depth probe into an unaffected area of the medial tibial plateau 122 (i.e., an area of the plateau 122 that does not exhibit cartilage loss). The difference between the two depth measurements reflects the amount of cartilage loss on the medial tibial plateau 122. The surgeon then repeats the process on the lateral tibial plateau 124. Specifically, the surgeon inserts the graduated depth probe into both the affected area of the lateral tibial plateau 124 and an unaffected area of the lateral tibial plateau 124 with the difference between the two depth measurements reflecting the amount of cartilage loss on the lateral tibial plateau 124.

Once the surgeon has determined the amount of cartilage loss (if any) on both tibial plateaus 122, 124, the surgeon utilizes the tibial stylus instrument 150 to accommodate for such cartilage loss by adjusting the resection level of the side or sides of the patient's tibia 120 experiencing cartilage loss. For instance, if a surgeon preoperatively planned a 9 mm resection level (based on the planned use of a tibial construct having a 4 mm thick tibial tray and a 5 mm thick tibial bearing), but then intraoperatively determined that, for example, both of the patient's tibial plateaus 122, 124 exhibited 2 mm of cartilage loss, the surgeon sets the control knobs 168, 178 of the medial and lateral position adjustment assemblies 162, 164, respectively, to the “7” resection level indicator 180 to reduce the resection level by the measured amount of cartilage loss (e.g., 2 mm in the example described herein). By reducing the resection level to accommodate for the measured amount of cartilage loss, the tibial prosthetic may be implanted at a height which mimics the patient's native anatomy (as it was prior to the onset of cartilage loss). Once the surgeon has adjusted the settings of the tibial stylus instrument 150 to accommodate for the amount of measured cartilage loss, the surgeon lowers the height of the tibial alignment jig 10 until the pointer 158 formed in the posterior end of the medial stylus 154 rests on the lowest point (i.e., the dwell point) of the medial tibial plateau 122 of the patient's tibia 120 and the pointer 160 formed in the posterior end of the lateral stylus 156 rests on the lowest point (i.e., the dwell point) of the lateral tibial plateau 124 of the patient's tibia 120 and completes the positioning of the tibial cutting block 112 in the manner described above.

Whether by use in the instruments'mechanical alignment mode of operation or its kinematic alignment mode of operation, once the surgeon has positioned the cutting slot 114 of the tibial cutting block 112 in the desired (1) resection height, (2) varus/valgus cutting angle 118, and (3) posterior slope cutting angle 138, the surgeon may pin the tibial cutting block 112 to the proximal end 126 of the patient's tibia 120 by installing a pair of bone pins (not shown) through a selected pair of the block's pin holes. With the tibial cutting block 112 installed on the proximal end 126 of the patient's tibia 120, the surgeon removes the tibial stylus instrument 150 by sliding its mounting flange 152 out of the block's cutting slot 114. The surgeon may then use the installed tibial cutting block 112 to perform a proximal resection of the proximal end 126 of the patient's tibia 120 by advancing a bone saw blade (not shown) of a surgical saw through the cutting slot 114 to engage the patient's tibia 120 and operate the surgical saw to surgically form a planar resected surface of the patient's tibia 120. The surgeon may then perform the remaining surgical steps to complete the orthopaedic knee procedure.

It should be appreciated that use of the tibial alignment jig 10 and the tibial stylus instrument 150 in combination has been described herein and such a combination has significant advantages. However, certain of such advantages may be achieved by use of the two instruments separate from one another. For example, the tibial alignment jig 10 may be used with a traditional (e.g., single stylus) stylus instrument. Similarly, the tibial stylus instrument 150 may be used with traditional tibial alignment jigs.

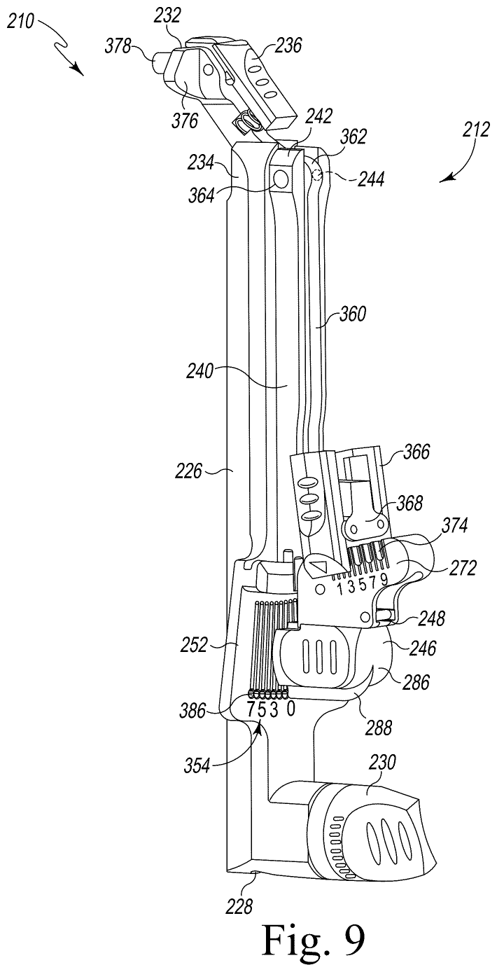

Referring to FIGS. 9-12, in another embodiment, an orthopaedic surgical instrument 210—in the form of a tibial alignment jig for use in the surgical preparation of a patient's tibia during performance of an orthopaedic knee procedure is shown. The tibial alignment jig 210 of FIGS. 9-12 is substantially similar to the tibial alignment jig 10 shown in FIGS. 1-3 and 5-7 and described herein. Accordingly, similar reference numbers are used in the description of the tibial alignment jig 210 to indicate features that are common between the tibial alignment jig 10 and the tibial alignment jig 210. The description of the tibial alignment jig 10 is incorporated by reference to apply to the tibial alignment jig 210, except in instances when it conflicts with the specific description and the drawings of the tibial alignment jig 210.

The tibial alignment jig 210 includes a proximal tibial jig assembly 212 and the distal tibial jig assembly 14. It should be appreciated that the distal tibial jig assembly 14 is removed from FIGS. 9-12.

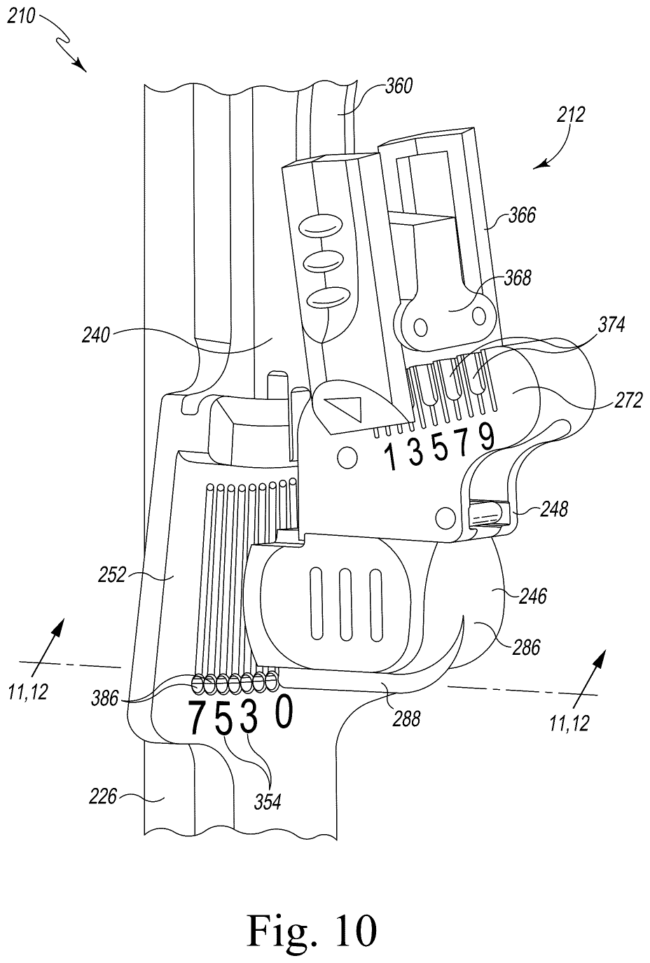

As can be seen in FIGS. 9 and 10, the proximal tibial jig assembly 212 includes a hollow extramedullary proximal rod 226. The proximal rod 226 has an elongated bore 228 formed therein that is configured to receive the alignment rod 22 of the distal tibial jig assembly 14. A locking knob 230 is used to lock the position of the alignment rod 22 (and hence the distal tibial jig assembly 14) relative to the proximal rod 226 (and hence the proximal tibial jig assembly 212). The locking knob 230 has an internal shaft that extends inwardly into the bore 228 of the proximal rod 226 and is displaced by rotation of the locking knob 230 to selectively clamp the alignment rod 22 of the distal tibial jig assembly 14 in a locked position within the proximal rod 226.

A block connector 232 is positioned on a superior end 234 of the proximal tibial jig assembly 212. In the illustrative embodiment described herein, the block connector 232 includes a mounting clip 236 and a pair of mounting pins 378, although other types of connectors are contemplated for use. The tibial cutting block 112 is clipped or otherwise secured to the proximal tibial jig assembly 212 by advancing the mounting pins 378 into a pair of corresponding holes in the cutting block 112 and then retaining the cutting block 112 against a clip body 376 with the mounting clip 236.

As can be seen in FIGS. 9 and 10, the proximal tibial jig assembly 212 includes a varus/valgus adjustment arm 240. A superior end 242 of the varus/valgus adjustment arm 240 is coupled to the block connector 232 and pivotally coupled to the proximal rod 226 via an anteroposteriorly extending pivot joint defined by an anteroposteriorly extending pivot pin 244. Rotation of the varus/valgus adjustment arm 240 about the pivot pin 244 adjusts the varus/valgus cutting angle 118 of the cutting slot 114 of the tibial cutting block 112. A locking grip 246 is secured to an inferior end 248 of the varus/valgus adjustment arm 240. The locking grip 246 is operable to lock the rotational position of the varus/valgus adjustment arm 240 (and hence the tibial cutting block 112 secured to the block connector 232) relative to the proximal rod 226.

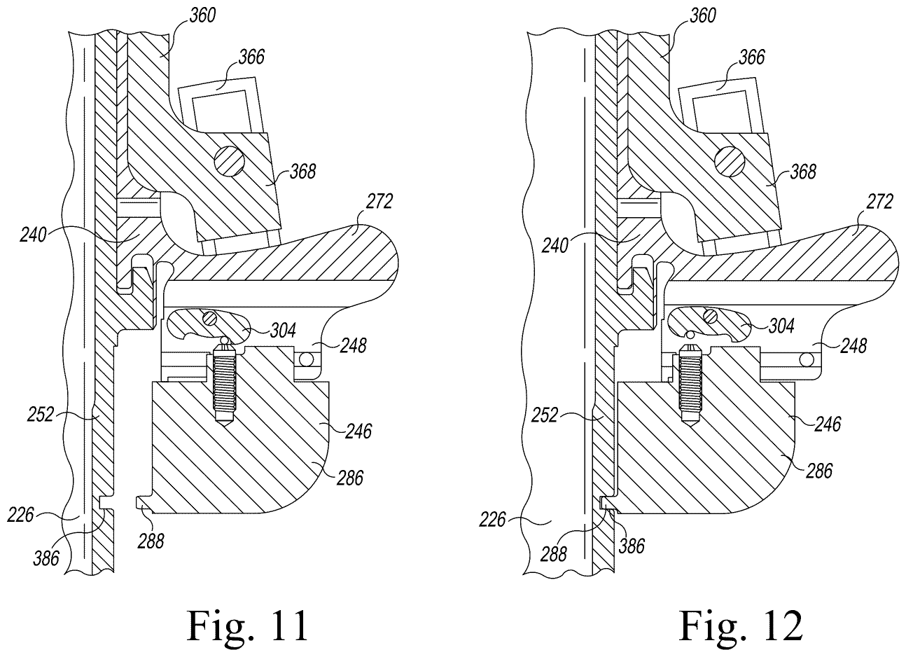

In particular, when the locking grip 246 is loosened (i.e., moved in an anterior direction), as shown in FIG. 11, the varus/valgus adjustment arm 240 is free to rotate relative to the proximal rod 226 about the anteroposteriorly extending pivot pin 244. Once a desired varus/valgus cutting angle 118 of the cutting slot 114 of the tibial cutting block 112 is achieved, the locking grip 246 may be moved in a posterior direction to lock the position of the varus/valgus adjustment arm 240 (and hence the tibial cutting block 112 secured to the block connector 232) relative to the proximal rod 226, as shown in FIG. 12.

The locking grip 246 includes a grip 286 and a pin 288 coupled to an inferior end of the grip 286, as shown in FIGS. 10-12. The grip 286 may be grasped by the surgeon and moved anteroposteriorly to lock and unlock the varus/valgus adjustment arm 240 relative to the proximal rod 226. The pin 288 acts as a position indicator of the varus/valgus adjustment arm 240. An indexing plate 252 is secured to the proximal rod 226 at a location superior to the locking knob 230.

The pin 288 moves radially along the indexing plate 252 during rotation of the varus/valgus adjustment arm 240 relative to proximal rod 226 about the anteroposteriorly extending pivot pin 244. The indexing plate 252 has a plurality of locking notches 386 radially positioned along its length. The pin 288 is received in one of the plurality of locking notches 386 to lock the position of the varus/valgus adjustment arm 240 relative to the proximal rod 226, as shown in FIG. 12.

The indexing plate 252 has indicia in the form of a plurality of angle indicators 354 radially engraved, printed, or otherwise formed thereon. Each of the plurality of angle indicators 354 represents a desired varus/valgus angle (e.g., left or right −0 degrees to 7 degrees) for selection by a surgeon during use of the tibial alignment jig 210.

When the grip 286 is grasped and pulled anteriorly relative to the indexing plate 252 such that the pin 288 is disengaged from the corresponding locking notch 386, as shown in FIG. 11, the varus/valgus adjustment arm 240 is free to rotate relative to the proximal rod 226. Once a desired varus/valgus angle of the cutting slot 114 of the tibial cutting block 112 is achieved, the grip 286 may be released or moved posteriorly so that the pin 288 reengages with a corresponding locking notch 386 to lock the position of the varus/valgus adjustment arm 240 (and hence the tibial cutting block 112 secured to the block connector 232) relative to the proximal rod 226, as shown in FIG. 12. As shown in FIGS. 11 and 12, the locking grip 246 includes a cam 304 that interacts with the grip 286 while the locking grip 246 is moved between its locked and unlocked positions. The cam 304 engages the grip 286 such that the locking grip 246 remains in its locked and unlocked positions until acted upon by the surgeon.

As can be seen in FIGS. 9 and 10, the proximal tibial jig assembly 212 includes a posterior slope adjustment arm 360. A superior end 362 of the posterior slope adjustment arm 360 is pivotally coupled to the varus/valgus adjustment arm 240 via a mediolaterally extending pivot joint defined by an mediolaterally extending pivot pin 364. Rotation of the posterior slope adjustment arm 360 about the pivot pin 364 adjusts the posterior slope angle of the cutting slot 114 of the tibial cutting block 112. Moreover, as shown in FIG. 9, by virtue of being coupled to the superior end 242 of the varus/valgus adjustment arm 240, the posterior slope adjustment arm 360 rotates with the varus/valgus adjustment arm 240 about the anteroposteriorly extending pivot pin 244. In other words, the posterior slope adjustment arm 360 rotates relative to the proximal rod 226 about both the anteroposteriorly extending pivot pin 244 and the mediolaterally extending pivot pin 364.

The superior end 362 of the posterior slope adjustment arm 360 is coupled to the block connector 232. In the illustrative example disclosed herein, the clip body 376 of the block connector 232 is integrally formed with the superior end 362 of the posterior slope adjustment arm 360. In such a way, the block connector 232 rotates relative to the proximal rod 226 about both the anteroposteriorly extending pivot pin 244 and the mediolaterally extending pivot pin 364.

A locking lever 366 is secured to an inferior end 368 of the posterior slope adjustment arm 360. The locking lever 366 is operable to lock the rotational position of the posterior slope adjustment arm 360 (and hence the tibial cutting block 112 secured to the block connector 232) relative to the varus/valgus adjustment arm 240. In particular, an indexing plate 272 extends outwardly from the varus/valgus adjustment arm 240 at a location near the locking grip 246. The indexing plate 272 has a plurality of locking notches 374 radially positioned along its length. Each of the plurality of locking notches 374 represents a desired posterior slope angle (e.g., 0 degrees to 10 degrees) for selection by a surgeon during use of the tibial alignment jig 210. The locking lever 366 is spring-loaded or otherwise urged in the direction of the locking notches 374. Thus, when the locking lever 366 is squeezed or otherwise disengaged from the locking notches 374, the posterior slope adjustment arm 360 is free to rotate relative to the varus/valgus adjustment arm 240 (and hence the proximal rod 226) about the mediolaterally extending pivot pin 364. Once a desired posterior slope cutting angle 138 of the cutting slot 114 of the tibial cutting block 112 is achieved, the locking lever 366 may be released or otherwise reengaged with the corresponding locking notch 374 to lock the position of the posterior slope adjustment arm 360 (and hence the tibial cutting block 112 secured to the block connector 232) relative to the varus/valgus adjustment arm 240 (and hence the proximal rod 226).

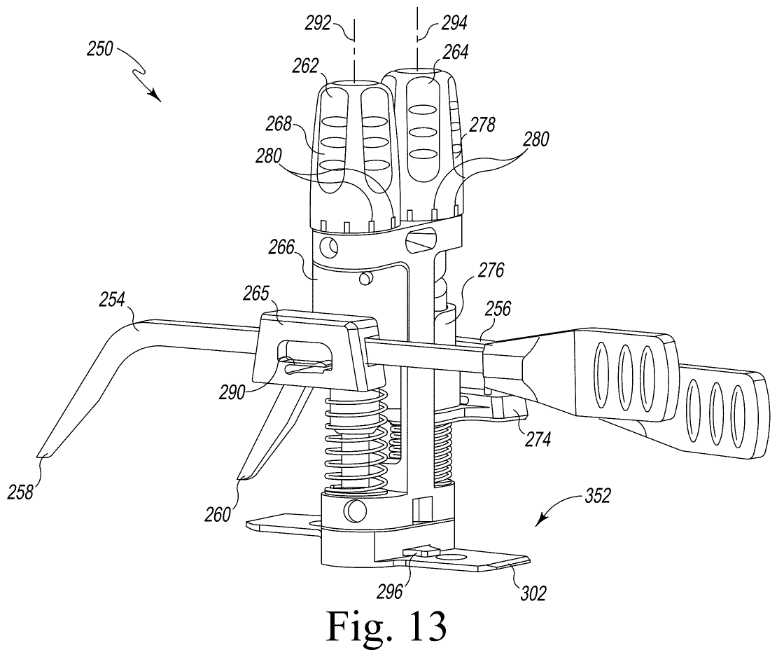

Referring to FIGS. 13-15, a tibial stylus instrument 250 is shown. The tibial stylus instrument 250 of FIGS. 13-15 is substantially similar to the tibial stylus instrument 150 shown in FIGS. 4, 7, and 8 and described herein. Accordingly, similar reference numbers are used in the description of the tibial stylus instrument 250 to indicate features that are common between the tibial stylus instrument 150 and the tibial stylus instrument 250. The description of the tibial stylus instrument 150 is incorporated by reference to apply to the tibial stylus instrument 250, except in instances when it conflicts with the specific description and the drawings of the tibial stylus instrument 250.

The tibial stylus instrument 250 includes a mounting flange 352 configured to be positioned in the cutting slot 114 of the tibial cutting block 112 so as to secure the tibial stylus instrument 250 to the cutting block 112. The tibial stylus instrument 250 also includes a pair of posteriorly extending styluses 254, 256 that are configured to contact the tibial plateaus 122, 124 of the patient's tibia 120. In particular, as shown in FIG. 13, the medial stylus 254 has a pointer 258 formed in its posterior end that is configured to rest on the medial tibial plateau 122 of the patient's tibia 120. Similarly, the lateral stylus 256 has a pointer 260 formed in its posterior end that is configured to rest on the lateral tibial plateau 124 of the patient's tibia 120.

The tibial stylus instrument 250 also includes a pair of position adjustment assemblies 262, 264 that are independently operable to upwardly and downwardly (i.e., superiorly and inferiorly) adjust the position of the styluses 254, 256 relative to the mounting flange 352 (and hence the cutting slot 114 when the instrument 250 is coupled to the cutting block 112), respectively. The medial position adjustment assembly 262 includes a housing 265 and a slider 266. The medial stylus 254 extends through the housing 265, which is secured to the slider 266. A control knob 268 is operable to move the slider 266 superoinferiorly to adjust the position of the medial stylus 254 relative to the mounting flange 352 (and hence the cutting slot 114 when the instrument 250 is coupled to the cutting block 112). In particular, rotation of the control knob 268 in a first direction (e.g., clockwise) moves the slider 266 upwardly thereby moving the medial stylus 254 upwardly (i.e., superiorly) in the direction away from the mounting flange 352 (and hence the cutting slot 114 when the instrument 250 is coupled to the cutting block 112). Conversely, rotation of the control knob 268 in a second, opposite direction (e.g., counterclockwise) moves the slider 266 downwardly thereby moving the medial stylus 254 downwardly (i.e., inferiorly) in the direction toward the mounting flange 352 (and hence the cutting slot 114 when the instrument 250 is coupled to the cutting block 112).

Similarly, the lateral position adjustment assembly 264 includes a housing 274 and a slider 276. The lateral stylus 256 extends through the housing 274, which is secured to the slider 276. A control knob 278 is operable to move the slider 276 superoinferiorly to adjust the position of the lateral stylus 256 relative to the mounting flange 352 (and hence the cutting slot 114 when the instrument 250 is coupled to the cutting block 112). In particular, rotation of the control knob 278 in a first direction (e.g., clockwise) moves the slider 276 upwardly thereby moving the lateral stylus 256 upwardly (i.e., superiorly) in the direction away from the mounting flange 352 (and hence the cutting slot 114 when the instrument 250 is coupled to the cutting block 112). Conversely, rotation of the control knob 278 in a second, opposite direction (e.g., counterclockwise) moves the slider 276 downwardly thereby moving the lateral stylus 256 downwardly (i.e., inferiorly) in the direction toward the mounting flange 352 (and hence the cutting slot 114 when the instrument 250 is coupled to the cutting block 112).

As can be seen in FIG. 13, the control knobs 268, 278 have indicia in the form of a plurality of resection level indicators 280 engraved, printed, or otherwise formed thereon. Each of the plurality of resection level indicators 280 corresponds to a resection amount, as measured in millimeters (e.g., 3 mm to 13 mm) for selection by a surgeon during use of the tibial stylus instrument 250. A surgeon may operate the position adjustment assemblies 262, 264 to change the height of the cutting slot 114 when the tibial stylus instrument 250 is coupled to the tibial cutting block 112. In particular, the surgeon may rotate one or both the control knobs 268, 278 to a desired resection level indicator 280 to adjust upwardly or downwardly the height of the block's cutting slot 114 on the patient's tibia 120. While doing so, the surgeon may also adjust the cutting angle of the block's cutting slot 114 by rotating the control knobs 268, 278 to different resection level indicators 280.

The surgeon may also move the medial stylus 254 anteroposteriorly relative to the housing 265. The housing 265 includes a spring 290 therein, as shown in FIG. 13. The spring 290 acts against the medial stylus 254 to maintain a position of the medial stylus 254 relative to the housing 265 unless or until acted upon by the surgeon.

The housing 265, and thus the medial stylus 254, can rotate relative to the slider 266 about an axis 292 extending superoinferiorly through the position adjustment assembly 262. In this way, the pointer 258 of the medial stylus 254 can move mediolaterally toward and away from the lateral stylus 256.

The surgeon may also move the lateral stylus 256 anteroposteriorly relative to the housing 274. The housing 274 includes a spring therein (not shown, but similar to the spring 290). The spring acts against the lateral stylus 256 to maintain a position of the lateral stylus 256 relative to the housing 274 unless or until acted upon by the surgeon.

The housing 274, and thus the lateral stylus 256, can rotate relative to the slider 276 about an axis 294 extending superoinferiorly through the position adjustment assembly 264. In this way, the pointer 260 of the lateral stylus 256 can move mediolaterally toward and away from the medial stylus 254.

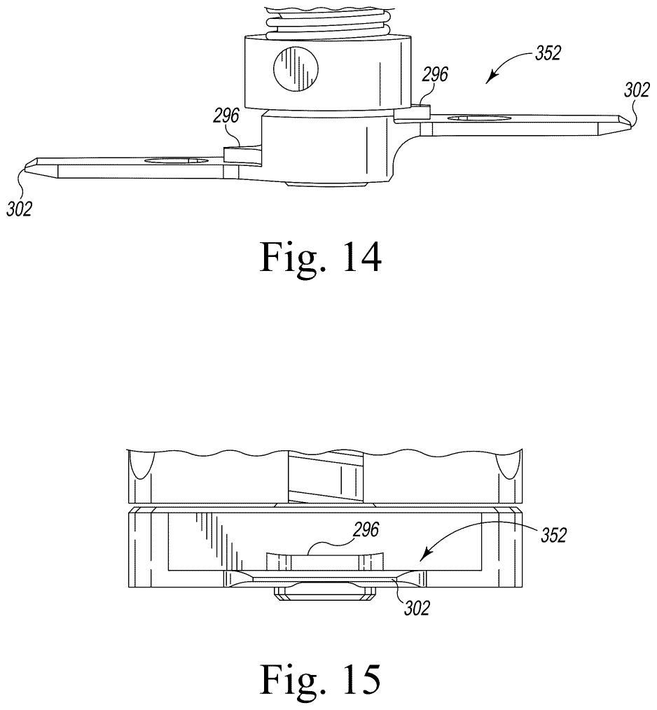

As shown in FIGS. 14 and 15, two locating tabs 296 extend superiorly from the mounting flange 352. One of the locating tabs 296 is configured to fit within a locating slot 298 of the cutting block 112 when the instrument 250 is coupled to the cutting block 112 (see FIG. 8). In particular, the locating tab 296 and the locating slot 298 are similarly sized and shaped such that the locating tab 296 is snugly received into engagement within the locating slot 298. This engagement between the locating tab 296 and the locating slot 298 helps to ensure proper alignment of the instrument 250 and the cutting block 112 when the instrument 250 is coupled to the cutting block 112.

As shown in FIGS. 14 and 15, the mounting flange 352 defines chamfered edges 302 on opposing ends thereof. One of the chamfered edges 302 is inserted into the cutting slot 114 of the cutting block 112. The chamfered edge 302 allows the mounting flange 352 to be inserted into the cutting slot 114. The chamfered edges 302 may also be referred to as tapered edges 302. As also shown in FIG. 15, the chamfered edges 302 have an arcuate shape along the mediolateral direction. The arcuate shape interacts with the cutting slot 114 of the cutting block 112 to form a friction fit between the instrument 250 and the cutting block 112 thereby maintaining a position of the instrument 250 relative to the cutting block 112.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic surgical instrument assembly for resecting a patient's tibia during an orthopaedic knee replacement procedure, comprising:
a tibial cutting block having a mediolaterally extending cutting slot,
a proximal tibial jig assembly having (i) an extramedullary proximal rod, and (ii) a block connector on a superior end of the proximal tibial jig assembly, the tibial cutting block being secured to the proximal tibial jig assembly with the block connector, wherein (a) the block connector pivots relative to the proximal rod about a mediolaterally extending pivot joint so as to adjust a posterior slope angle of the cutting slot, and (b) the block connector pivots relative to the proximal rod about an anteroposteriorly extending pivot joint so as to adjust a varus/valgus angle of the cutting slot, and
a distal tibial jig assembly secured to the proximal tibial jig assembly, the distal tibial jig assembly having an ankle clamp positioned on its inferior end.

2. The orthopaedic surgical instrument assembly of claim 1, wherein:
the proximal tibial jig assembly further comprises a varus/valgus adjustment arm,
a superior end of the varus/valgus adjustment arm is coupled to the block connector and pivotally coupled to the proximal rod via the anteroposteriorly extending pivot joint, and
rotation of the varus/valgus adjustment arm about the anteroposteriorly extending pivot joint adjusts the varus/valgus angle of the cutting slot.

3. The orthopaedic surgical instrument assembly of claim 2, wherein:
an inferior end of the varus/valgus adjustment arm includes a locking grip, and
the locking grip is operable to lock the varus/valgus adjustment arm in a selected rotational position relative to the proximal rod.

4. The orthopaedic surgical instrument assembly of claim 3, wherein the proximal tibial jig assembly further comprises an indexing plate coupled to the proximal rod, the indexing plate having a plurality of locking notches positioned along its length.

5. The orthopaedic surgical instrument assembly of claim 4, wherein the locking grip of the varus/valgus adjustment arm engages one of the plurality of locking notches of the indexing plate to lock the varus/valgus adjustment arm in the selected rotational position relative to the proximal rod, and wherein the locking grip includes a grip and a cam that interacts with the grip to maintain the grip in its locked or unlocked position.

6. The orthopaedic surgical instrument assembly of claim 2, wherein:
the proximal tibial jig assembly further comprises a posterior slope adjustment arm,
a superior end of the posterior adjustment arm is coupled to the block connector and pivotally coupled to the varus/valgus adjustment arm via the mediolaterally extending pivot joint, and
rotation of the posterior slope adjustment arm about the mediolaterally extending pivot joint adjusts the posterior slope angle of the cutting slot.

7. The orthopaedic surgical instrument assembly of claim 6, wherein:
an inferior end of the posterior slope adjustment arm includes a locking lever, and
the locking lever is operable to lock the posterior slope adjustment arm in a selected rotational position relative to the proximal rod.

8. The orthopaedic surgical instrument assembly of claim 6, wherein the block connector is integrally formed in the superior end of the posterior slope adjustment arm.

9. The orthopaedic surgical instrument assembly of claim 1, wherein the block connector comprises a locking clip.

10. A method of surgically preparing a patient's tibia during an orthopaedic surgical knee procedure, comprising:
securing a tibial cutting block to a proximal tibial jig assembly, the tibial cutting block having a mediolaterally extending cutting slot,
securing a distal tibial jig assembly to the proximal tibial jig assembly, the distal tibial jig assembly having an ankle clamp,
rotating a varus/valgus adjustment arm of the proximal tibial jig assembly so as to adjust a varus/valgus angle of the cutting slot, and
rotating a posterior slope adjustment arm of the proximal tibial jig assembly so as to adjust a posterior slope angle of the cutting slot.

11. The method of claim 10, wherein rotating the varus/valgus adjustment arm comprises:
rotating the varus/valgus adjustment arm of the proximal tibial jig assembly so as to position the varus/valgus adjustment arm in a selected rotational position, and
operating a locking grip to lock the varus/valgus adjustment arm in the selection rotational position.

12. The method of claim 10, wherein rotating the posterior slope adjustment arm comprises:
rotating the posterior slope adjustment arm of the proximal tibial jig assembly so as to position the posterior slope adjustment arm in a selected rotational position, and
operating a locking lever to lock the posterior slope adjustment arm in the selection rotational position.

13. The method of claim 10, wherein rotating the posterior slope adjustment arm of the proximal tibial jig assembly so as to adjust the posterior slope angle of the cutting slot comprises pivoting the posterior slope adjustment arm relative to the varus/valgus adjustment arm.

14. The method of claim 10, further comprising positioning the assembled proximal and distal tibial jig assemblies on the patient's tibia such that the ankle clamp is clamped to the patient's ankle and the tibial cutting block abuts the patient's proximal tibia.

15. An orthopaedic surgical instrument assembly for resecting a patient's tibia during an orthopaedic knee replacement procedure, comprising:
a tibial cutting block having a mediolaterally extending cutting slot,
a proximal tibial jig assembly having (i) an extramedullary proximal rod, (ii) a varus/valgus adjustment arm pivotally coupled to the proximal rod via an anteroposteriorly extending pivot joint, and (iii) a posterior slope adjustment arm pivotally coupled to the varus/valgus adjustment arm via a mediolaterally extending pivot joint, and (iv) a block connector secured to a superior end of the posterior slope adjustment arm, wherein (a) the tibial cutting block is configured to be secured to the block connector, (b) rotation of the varus/valgus adjustment arm about the anteroposteriorly extending pivot joint adjusts a varus/valgus angle of the cutting slot, and (c) rotation of the posterior slope adjustment arm about the mediolaterally extending pivot joint adjusts a posterior slope angle of the cutting slot, and
a distal tibial jig assembly configured to be secured to the proximal tibial jig assembly, the distal tibial jig assembly having an ankle clamp positioned on its inferior end.

16. The orthopaedic surgical instrument assembly of claim 15, wherein:
an inferior end of the varus/valgus adjustment arm includes a locking grip, and
the locking grip is operable to lock the varus/valgus adjustment arm in a selected rotational position relative to the proximal rod.

17. The orthopaedic surgical instrument assembly of claim 16, wherein the proximal tibial jig assembly further comprises an indexing plate coupled to the proximal rod, the indexing plate having a plurality of locking notches positioned along its length, and wherein the locking grip of the varus/valgus adjustment arm engages one of the plurality of locking notches of the indexing plate to lock the varus/valgus adjustment arm in the selected rotational position relative to the proximal rod.

18. The orthopaedic surgical instrument assembly of claim 15, wherein:
an inferior end of the posterior slope adjustment arm includes a locking lever, and
the locking lever is operable to lock the posterior slope adjustment arm in a selected rotational position relative to the proximal rod.

19. The orthopaedic surgical instrument assembly of claim 15, wherein the block connector is integrally formed in the superior end of the posterior slope adjustment arm.

20. The orthopaedic surgical instrument assembly of claim 15, wherein the block connector comprises a locking clip.
